(19) United States
(12) Patent Application Publication
(10) Pub. No.: US 2024/0150500 A1
(43) Pub. Date: May 9, 2024

(54) SUBSTRATE PROCESSING APPARATUS

(71) Applicant: TOKYO ELECTRON LIMITED, Tokyo (JP)

(72) Inventors: Tatsuya YAMAGUCHI, Nirasaki City (JP); Syuji NOZAWA, Nirasaki City (JP)

(21) Appl. No.: 18/500,087

(22) Filed: Nov. 1, 2023

(30) Foreign Application Priority Data
Nov. 9, 2022 (JP) 2022-179529

(51) Int. Cl. C08F 2/34; C08F 2/00; C08G 85/00

(52) U.S. Cl. CPC C08F 2/34; C08F 2/008; C08G 85/008

(57) ABSTRACT

A substrate processing apparatus includes a processing container; a stage on which a substrate is placed, the stage being provided in the processing container; a gas supply provided at a position facing the stage and configured to supply a first processing gas containing a first monomer and a second processing gas containing a second monomer into the processing container to form a film of a polymer on the substrate; and a driver configured to move the stage so as to change a distance between the gas supply and the stage. The gas supply is configured to supply the first processing gas and the second processing gas into a space between the gas supply and the stage from an outside of a region on the stage in which the substrate is placed, when viewed in a direction from the gas supply toward the stage.

### CROSS-REFERENCE TO RELATED APPLICATION

This application is based upon and claims the benefit of priority from Japanese Patent Application No. 2022-179529, filed on Nov. 9, 2022, the entire contents of which are incorporated herein by reference.

### TECHNICAL FIELD

The present disclosure relates to a substrate processing apparatus.

### BACKGROUND

In the related art, there is known a vapor deposition polymerization apparatus in which two types of raw material monomer gases flow into a processing chamber that accommodates a substrate to form a polymer film on the surface of the substrate by vapor deposition polymerization. This vapor deposition polymerization apparatus includes a shower plate provided on the surface of the processing chamber facing the substrate, and a gas introduction chamber adjacent to the processing chamber with the shower plate interposed therebetween. The gas introduction chamber is provided with a partition wall that partitions the internal space of the gas introduction chamber into two gas introduction passages for individually supplying one and the other of the two types of raw material monomer gases. The partition wall is formed so that the two gas introduction passages are arranged alternately at a predetermined pitch along the plate surface of the shower plate. A large number of blow-off holes are formed in the shower plate along each of the gas introduction passages.

### PRIOR ART DOCUMENT

### Patent Document

Patent Document 1: Japanese Patent Laid-Open Publication No. 2011-117030

### SUMMARY

According to one embodiment of the present disclosure, there is provided a substrate processing apparatus including: a processing container; a stage on which a substrate is placed, the stage being provided in the processing container; a gas supply provided at a position facing the stage and configured to supply a first processing gas containing a first monomer and a second processing gas containing a second monomer into the processing container to form a film of a polymer composed of the first monomer and the second monomer on the substrate; and a driver configured to move the stage so as to change a distance between the gas supply and the stage, wherein the gas supply is configured to supply the first processing gas and the second processing gas into a space between the gas supply and the stage from an outside of a region on the stage in which the substrate is placed, when viewed in a direction from the gas supply toward the stage.

### BRIEF DESCRIPTION OF DRAWINGS

The accompanying drawings, which are incorporated in and constitute a part of the specification, illustrate embodiments of the present disclosure, and together with the general description given above and the detailed description of the embodiments given below, serve to explain the principles of the present disclosure.

FIG. 1 is a schematic cross-sectional view showing an example of a substrate processing apparatus according to a first embodiment of the present disclosure.

FIG. 2 is a diagram showing an example of the arrangement of a diffusion chamber within a gas supply.

FIG. 3 is a schematic cross-sectional view showing an example of a substrate processing apparatus according to a comparative example.

FIG. 4 is a diagram showing an example of the relationship between the distance between a gas supply and a stage and the thickness distribution of an organic film formed on a substrate according to a comparative example.

FIG. 5 is a diagram showing an example of the relationship between the distance between a gas supply and a stage and the thickness distribution of an organic film formed on a substrate according to a first embodiment.

FIG. 6 is a diagram showing an example of the relationship between the distance between a gas supply and a stage and the uniformity of the thickness of an organic film formed on a substrate.

FIG. 7 is a schematic cross-sectional view showing another example of the substrate processing apparatus according to the first embodiment.

FIG. 8 is a schematic cross-sectional view showing an example of a substrate processing apparatus according to a second embodiment of the present disclosure.

FIG. 9 is an enlarged sectional view for explaining an example of the distance between a stage and a gas supply.

FIG. 10 is a diagram showing an example of the deposition rate and the uniformity of the thickness of an organic film.

### DETAILED DESCRIPTION

Reference will now be made in detail to various embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, systems, and components have not been described in detail so as not to unnecessarily obscure aspects of the various embodiments.

Embodiments of the disclosed substrate processing apparatus will now be described in detail based on the drawings. The disclosed substrate processing apparatus is not limited to the following embodiments.

Incidentally, in order to make uniform the thickness of a film formed on a substrate, a film-forming gas may be supplied in a shower form into a processing container. However, when the film-forming gas is exhausted from below or around a stage on which the substrate is placed, the concentration of the film-forming gas in the peripheral region of the substrate is lower than in the central region of the substrate. If there are regions on the substrate where the concentration of the film-forming gas is different, the depositions of the organic film in those regions may be different, and the uniformity of the thickness of the organic film formed over the entire substrate may be reduced.

Therefore, the present disclosure provides a technique capable of improving the uniformity of the thickness of an organic film formed on a substrate.

### First Embodiment

### Configuration of Substrate Processing Apparatus 10

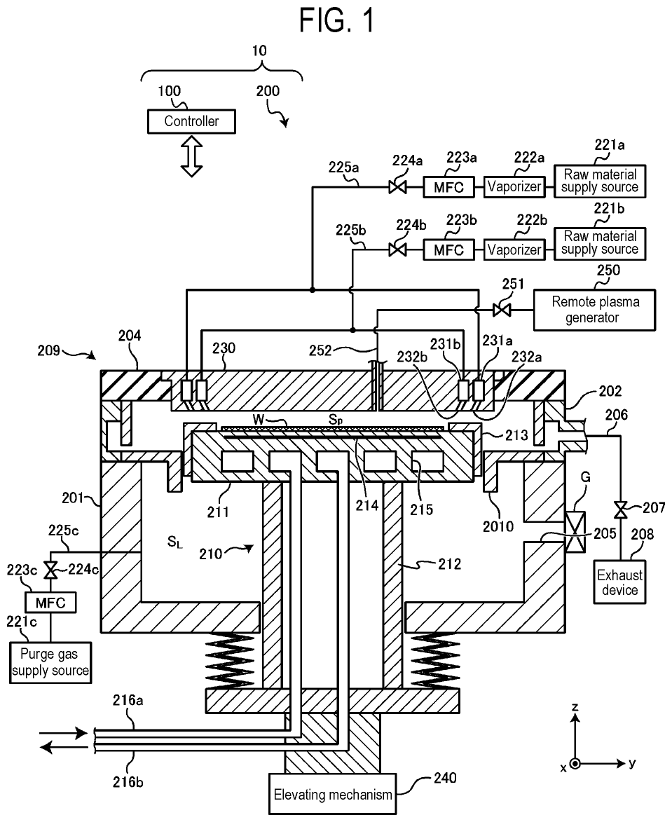

FIG. 1 is a schematic cross-sectional view showing an example of a substrate processing apparatus 10 according to a first embodiment of the present disclosure. The substrate processing apparatus 10 includes an apparatus main body 200 and a controller 100 that controls the apparatus main body 200. The apparatus main body 200 includes a processing container 209. The processing container 209 includes a lower container 201, an exhaust duct 202, a support structure 210, and a gas supply 230.

The lower container 201 is made of, for example, a metal such as aluminum or the like. The exhaust duct 202 has a hollow rectangular vertical cross section and extends annularly along the upper portion of the lower container 201. An exhaust blade 2010 having a cross-sectional shape that extends downward along the side wall of the lower container 201 is provided on the lower container 201 below the exhaust duct 202. The exhaust blade 2010 is formed in an annular shape along the side wall of the lower container 201.

Further, an annular insulating member 204 is arranged above the exhaust duct 202. The gas supply 230 is provided above the lower container 201 and supported by the insulating member 204. The support structure 210 on which a substrate W is placed is provided approximately at the center of the lower container 201. The space between the support structure 210 and the gas supply 230 is defined as a processing space S_{P}.

An opening 205 for loading and unloading the substrate W is formed in the side wall of the lower container 201. The opening 205 is opened and closed by a gate valve G.

One end of an exhaust pipe 206 is connected to the exhaust duct 202. The other end of the exhaust pipe 206 is connected to an exhaust device 208 including a vacuum pump or the like via a pressure regulation valve 207 such as an APC (Auto Pressure Controller) valve or the like. The pressure regulation valve 207 is controlled by the controller 100 to regulate the pressure within the processing space S_{P} to a preset pressure.

A heater (not shown) is provided on the side wall of the exhaust duct 202 and the upper surface of the gas supply 230. The exhaust duct 202 and the gas supply 230 are heated to a temperature of, for example, 200 degrees C. or higher. Thus, adhesion of reaction byproducts (so-called deposits) to the exhaust duct 202 and the gas supply 230 can be suppressed to some extent. The exhaust pipe 206, the pressure regulation valve 207, and the exhaust device 208 may also be provided with heaters and may be heated to a temperature at which deposits are difficult to adhere to them.

The support structure 210 includes a stage 211 and a support part 212. The stage 211 is made of, for example, a metal such as aluminum or the like, and the substrate W is placed on the upper surface thereof. The gas supply 230 is provided at a position facing stage 211. The support part 212 is made of, for example, a metal such as aluminum or the like, and is formed in a cylindrical shape so as to support the stage 211 from below.

An annular cover member 213 is provided on the peripheral edge of the upper surface of the stage 211 on which the substrate W is placed and on the side surface of the stage 211 so as to surround the peripheral edge of the upper surface of the stage 211 and the side surface of the stage 211.

A heater 214 is embedded in the stage 211. The heater 214 heats the substrate W placed on the stage 211 using the supplied power. The power supplied to heater 214 is controlled by the controller 100.

Further, a flow path 215 through which a coolant flows is formed inside the stage 211. A chiller unit (not shown) is connected to the flow path 215 via a pipe 216a and a pipe 216b. The coolant whose temperature has been adjusted to a predetermined temperature by the chiller unit is supplied to the flow path 215 via the pipe 216a. The coolant that has circulated within the flow path 215 is returned to the chiller unit via the pipe 216b. The stage 211 is cooled by the coolant circulating in the flow path 215. The chiller unit is controlled by the controller 100.

The support part 212 is arranged within the lower container 201 so as to pass through an opening formed at the bottom of the lower container 201. The support part 212 is moved up and down by the drive of the elevating mechanism 240. By driving the elevating mechanism 240, the distance between the stage 211 and the gas supply 230 is controlled. The elevating mechanism 240 is an example of a driver.

When loading the substrate W, the support structure 210 is lowered by driving the elevating mechanism 240, and the gate valve G is opened. Then, the substrate W is loaded into the lower container 201 through the opening 205 by a transfer robot (not shown), and is delivered to lift pins (not shown) protruding above the stage 211. Then, the substrate W is placed on the stage 211 by lowering the lift pins (not shown). Then, the gate valve G is closed, the support structure 210 is raised by driving the elevating mechanism 240, and a film-forming process on the substrate W is performed.

Moreover, when unloading the substrate W, the support structure 210 is lowered by driving the elevating mechanism 240, and the gate valve G is opened. Then, the substrate W is lifted from the stage 211 by raising the lift pins (not shown). Then, the substrate W on the lift pins (not shown) is unloaded from the lower container 201 through the opening 205 by the transfer robot (not shown).

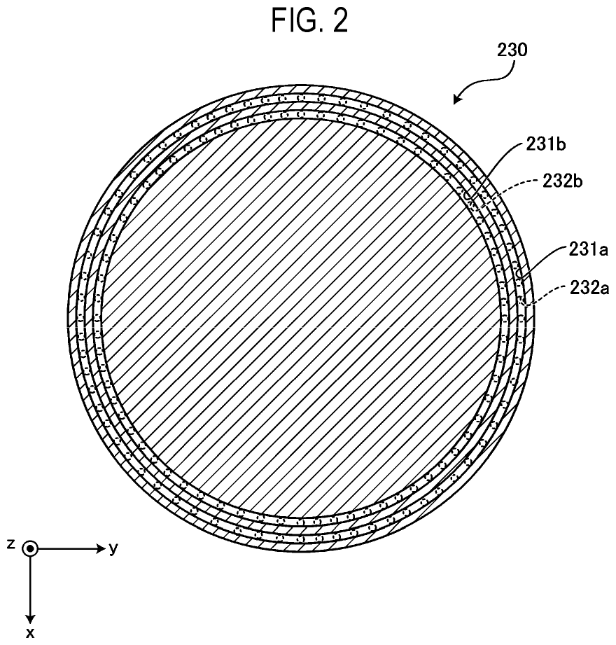

The gas supply 230 includes a diffusion chamber 231a and a diffusion chamber 231b. In this embodiment, for example, as shown in FIG. 2, the diffusion chamber 231a and the diffusion chamber 231b are formed in an annular shape along the outer wall of the gas supply 230. FIG. 2 shows an example of the arrangement of the diffusion chamber 231a and the diffusion chamber 231b when the gas supply 230 is viewed in the direction from the gas supply 230 toward the stage 211 (e.g., along the z-axis). The diffusion chamber 231a and the diffusion chamber 231b do not communicate with each other and form independent spaces.

Returning to FIG. 1, the description will be continued. A pipe 225a is connected to the diffusion chamber 231a, and a pipe 225b is connected to the diffusion chamber 231b. The pipe 225a is an example of a first pipe, and the pipe 225b is an example of a second pipe.

A valve 224a, an MFC (Mass Flow Controller) 223a, a vaporizer 222a, and a raw material supply source 221a are connected to the pipe 225a. The raw material supply source 221a is a supply source of isocyanate, which is an example of a first monomer. The vaporizer 222a vaporizes the isocyanate liquid supplied from the raw material supply source 221a. The MFC 223a controls the flow rate of isocyanate vapor vaporized by the vaporizer 222a. The valve 224a controls the supply and stop of the isocyanate vapor with respect to the pipe 225a.

A valve 224b, an MFC 223b, a vaporizer 222b, and a raw material supply source 221b are connected to the pipe 225b. The raw material supply source 221b is a supply source of amine, which is an example of a second monomer. The vaporizer 222b vaporizes the amine liquid supplied from the raw material supply source 221b. The MFC 223b controls the flow rate of amine vapor vaporized by the vaporizer 222b. The valve 224b controls the supply and stop of amine vapor with respect to the pipe 225b. A gas containing isocyanate vapor is an example of a first processing gas, and a gas containing amine vapor is an example of a second processing gas.

For example, as shown in FIGS. 1 and 2, a plurality of discharge ports 232a is formed at the bottom of the diffusion chamber 231a, and a plurality of discharge ports 232b is formed at the bottom of the diffusion chamber 231b. The isocyanate vapor supplied into the diffusion chamber 231a through the pipe 225a is diffused within the diffusion chamber 231a and is discharged in a shower form into the processing space S_{P} through the respective discharge ports 232a. Further, the amine vapor supplied into the diffusion chamber 231b via the pipe 225b is diffused within the diffusion chamber 231b and is discharged in a shower form into the processing space S_{P} through the discharge ports 232b. The discharge ports 232a are an example of first discharge ports, and the discharge ports 232b are an example of second discharge ports. After the isocyanate vapor and the amine vapor are discharged into the processing space S_{P} through the discharge ports 232a and 232b, they are mixed in the processing space S_{P} to form an organic film of a polymer having urea bonds on the surface of the substrate W placed on the stage 211.

For example, a linear polyurea can be produced by using diisocyanate as the first monomer and diamine (e.g., primary amine) as the second monomer. The combination of diisocyanate and diamine is, for example, a combination of 4,4′-diphenylmethane diisocyanate (MDI) and 1,12-diaminododecane (DAD). The combination of diisocyanate and diamine is, for example, a combination of 1,3-bis(isocyanatomethyl)cyclohexane (H6XDI) and 1,12-diaminododecane (DAD). The combination of diisocyanate and diamine is, for example, a combination of 1,3-bis(isocyanatomethyl)cyclohexane (H6XDI) and 1,3-bis(aminomethyl)cyclohexane (H6XDA). The combination of diisocyanate and diamine is, for example, a combination of 1,3-bis(isocyanatomethyl)cyclohexane (H6XDI) and hexamethylene diamine (HMDA). The combination of diisocyanate and diamine is, for example, a combination of m-xylylene diisocyanate (XDI) and m-xylylene diamine (XDA). The combination of diisocyanate and diamine is, for example, a combination of m-xylylene diisocyanate (XDI) and benzylamine (BA).

For example, a crosslinking polyurea can be produced by using diisocyanate as the first monomer and triamine (e.g., primary amine) or a tetraamine (e.g., secondary amine) as the second monomer. Further, a trimer having a urea bond can be produced by using monoisocyanate as the first monomer and diamine (e.g., primary amine) as the second monomer. Further, a dimer having a urea bond can be produced by using monoisocyanate as the first monomer and monoamine (e.g., primary amine) as the second monomer.

The conductance of each discharge port 232a is smaller than the conductance of the diffusion chamber 231a. Thus, the isocyanate vapor supplied to the diffusion chamber 231a is sufficiently diffused within the diffusion chamber 231a, and then supplied into the processing space S_{P} from each discharge port 232a. Therefore, the isocyanate vapor is almost uniformly supplied into the processing space S_{P} along the diffusion chamber 231a from the outside of a region of the stage 211 in which the substrate W is placed.

Regarding each discharge port 232b, the conductance of each discharge port 232b is also smaller than the conductance of the diffusion chamber 231b. Thus, the amine vapor supplied to the diffusion chamber 231b is sufficiently diffused within the diffusion chamber 231b, and then supplied into the processing space S_{P} from each discharge port 232b. Therefore, the amine vapor is almost uniformly supplied into the processing space S_{P} along the diffusion chamber 231b from the outside of the region of the stage 211 in which the substrate W is placed.

In this embodiment, when viewed in the direction from the gas supply 230 toward the stage 211, the respective discharge ports 232a and 232b discharge gases obliquely inward from the outside of the region of the stage 211 in which the substrate W is placed. However, the disclosed technique is not limited thereto. As another example, the respective discharge ports 232a and 232b may discharge gases in a direction from the gas supply 230 toward the stage 211 (e.g., along the z-axis) or in a direction along the lower surface of the gas supply 230 (e.g., along the xy plane).

Further, a remote plasma generator 250 is connected to the gas supply 230 via a pipe 252 and a valve 251. The remote plasma generator 250 converts a gas such as an oxygen gas or the like into plasma and generates active species contained in the plasma. The valve 251 controls the supply and stop of active species generated by the remote plasma generator 250 with respect to the pipe 252. The active species supplied to the pipe 252 are supplied into the processing space S_{P}. As a result, the inside of the processing container 209 is cleaned. The remote plasma generator 250, the valve 251, and the pipe 252 are an example of a cleaner.

A valve 224c, an MFC 223c, and a purge gas supply source 221c are connected to the lower container 201 below the stage 211 via a pipe 225c. The purge gas supply source 221c is a supply source of a purge gas. The purge gas is, for example, an inert gas such as a nitrogen gas or a rare gas. The MFC 223c controls the flow rate of the purge gas supplied from the purge gas supply source 221c. The valve 224c controls the supply and stop of the purge gas with respect to the pipe 225c. The space within the lower container 201 below the stage 211 is defined as a lower space S_{L}. By supplying the purge gas into the lower space S_{L}, it is possible to suppress the gas supplied into the processing space S_{P} from entering the lower space S_{L}. Thus, it is possible to suppress the formation of reaction by-products on the inner wall of the lower container 201 due to the gas supplied into the processing space S_{P} entering the lower space S_{L}.

The controller 100 includes a memory, a processor, and an input/output interface. The memory stores a control program, a process recipe, and the like. The processor reads the control program from the memory and executes the same, and controls each part of the apparatus main body 200 via the input/output interface based on the recipe stored in the memory.

### Configuration of Substrate Processing Apparatus 10 according to Comparative Example

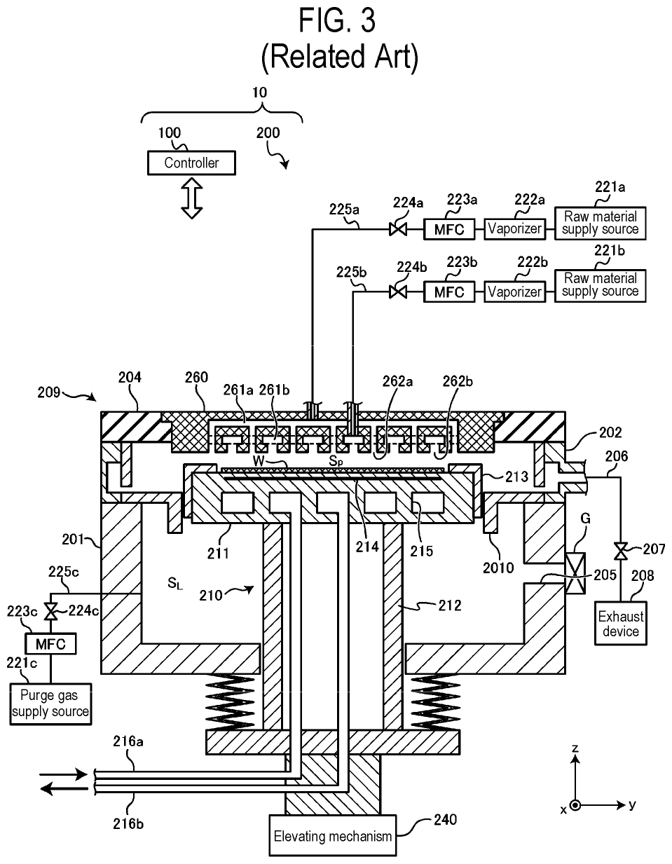

In order to demonstrate the effects of the substrate processing apparatus 10 according to this embodiment, a substrate processing apparatus 10 having a structure shown in, for example, FIG. 3 is used as a comparative example. The substrate processing apparatus 10 shown in FIG. 3 includes a gas supply 260 in place of the gas supply 230 of the substrate processing apparatus 10 according to this embodiment illustrated in FIG. 1. In the substrate processing apparatus 10 illustrated in FIG. 3, the members designated by the same reference numerals as those in the substrate processing apparatus 10 illustrated in FIG. 1 have the same functions as the members in the substrate processing apparatus 10 illustrated in FIG. 1. Therefore, the duplicate explanations thereof will be omitted.

A disc-shaped diffusion chamber 261a and a disc-shaped diffusion chamber 261b are formed inside the gas supply 260 of the substrate processing apparatus 10 according to the comparative example. The diffusion chamber 261a and the diffusion chamber 261b do not communicate with each other and form independent spaces. A pipe 225a is connected to the diffusion chamber 261a, and a pipe 225b is connected to the diffusion chamber 261b.

Furthermore, a plurality of discharge ports 262a and a plurality of discharge ports 262b are formed on the lower surface of the gas supply 260. Each discharge port 262a communicates with the diffusion chamber 261a, and each discharge port 262b communicates with the diffusion chamber 261b. The isocyanate vapor supplied into the diffusion chamber 261a via the pipe 225a is diffused within the diffusion chamber 261a, and is supplied in a shower form to the region of the stage 211 in which the substrate W is placed and the outside of the region of the stage 211 through the respective discharge ports 262a. Further, the amine vapor supplied into the diffusion chamber 261b via the pipe 225b is diffused within the diffusion chamber 261b, and is supplied in a shower form to the region of the stage 211 in which the substrate W is placed and the outside of the region of the stage 211 through the respective discharge ports 262b.

### Relationship between Distance between Gas supply and Stage and Film Thickness Distribution

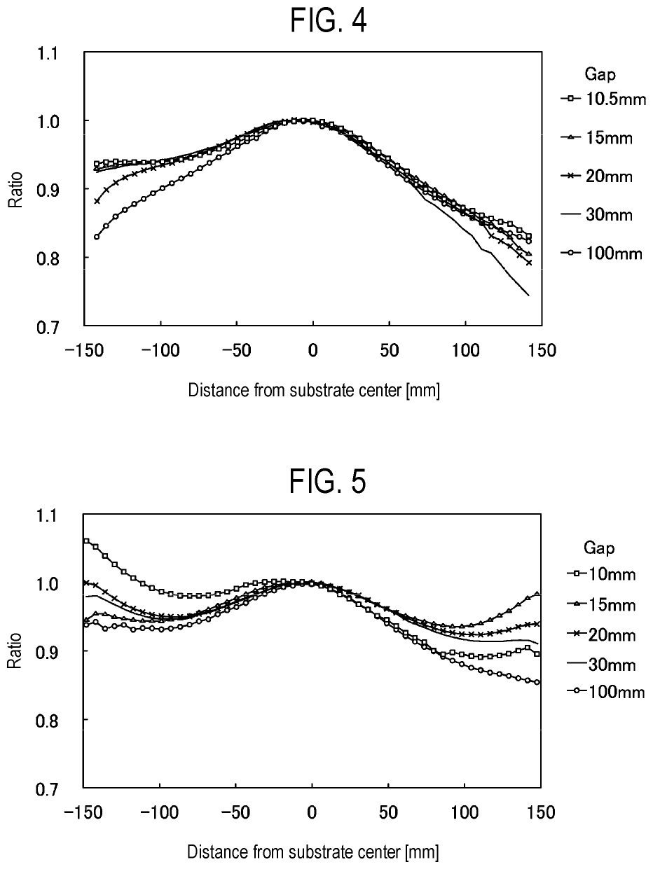

FIG. 4 is a diagram showing an example of the relationship between the distance between the gas supply 260 and the stage 211 and the thickness distribution of the organic film formed on the substrate W according to the comparative example. In FIG. 4, the distance between the gas supply 260 and the stage 211 is expressed as Gap. Further, in FIG. 4, the thickness of the organic film at each position on the substrate W is indicated as a relative value with respect to the thickness of the organic film at the center of the substrate W.

In the comparative example, as shown in FIG. 4, for example, even if the distance between the gas supply 260 and the stage 211 is changed, the tendency of the thickness distribution of the organic film formed on the substrate W does not change much. In the comparative example, while the gas is supplied substantially uniformly to the entire substrate W, the gas is exhausted from around the stage. Therefore, the gas concentration near the edge of the substrate W is lower than that near the center of the substrate W. When the concentration of the gas decreases, the deposition rate of the organic film decreases, and the thickness of the organic film formed on the substrate W decreases. Therefore, the thickness of the organic film near the edge of the substrate W is smaller than that at the center of the substrate W. This tendency is considered to remain unchanged even if the distance between the gas supply 260 and the stage 211 is changed.

FIG. 5 is a diagram showing an example of the relationship between the distance between the gas supply 230 and the stage 211 and the thickness distribution of the organic film formed on the substrate W according to the first embodiment. In FIG. 5, the distance between the gas supply 230 and the stage 211 is expressed as GAP. Further, in FIG. 5, the thickness of the organic film at each position on the substrate W is indicated as a relative value with respect to the thickness of the organic film at the center of the substrate W.

In this embodiment, when the distance between the gas supply 230 and the stage 211 is changed, the tendency of the thickness distribution of the organic film formed on the substrate W is changed more significantly than in the comparative example. In this embodiment, the gas is supplied into the processing space S_{P} from the outside of the region of the substrate W, and is exhausted from a region around the stage. When the distance between the gas supply 230 and the stage 211 is short, the conductance between the gas supply 230 and the stage 211 becomes small, and the gas supplied into the processing space S_{P} is difficult to reach the vicinity of the center of the substrate W. Therefore, the gas concentration near the center of the substrate W is lower than that near the edge of the substrate W, and the deposition rate of the organic film near the center of the substrate W is lower than that near the edge of the substrate W. As a result, it is considered that when the distance between the gas supply 230 and the stage 211 is short, the thickness of the organic film near the center of the substrate W is smaller than that near the edge of the substrate W.

On the other hand, when the distance between the gas supply 230 and the stage 211 is long, the conductance between the gas supply 230 and the stage 211 increases, and the gas supplied into the processing space S_{P} can sufficiently reach the vicinity of the center of the substrate W. Since the gas is exhausted from the region around the stage, the gas concentration near the edge of the substrate W is lower than that near the center of the substrate W. The deposition rate near the edge of the substrate W is lower than that near the center of the substrate W. As a result, when the distance between the gas supply 230 and the stage 211 is long, it is considered that the thickness of the organic film near the edge of the substrate W is smaller than that near the center of the substrate W.

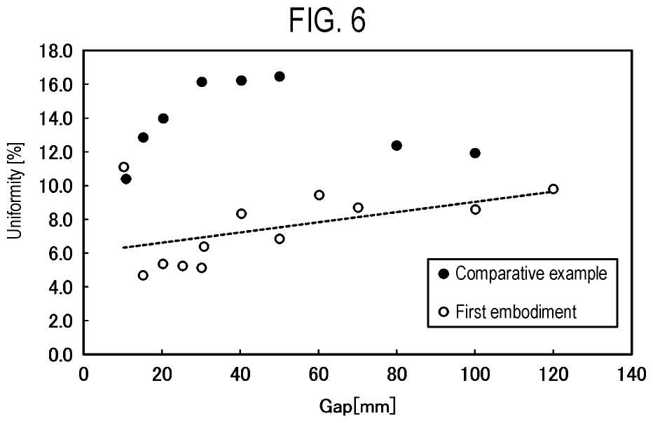

FIG. 6 is a diagram showing an example of the relationship between the Gap and the uniformity of the thickness of the organic film formed on the substrate W. Regarding the uniformity in FIG. 6, the smaller value means better uniformity. As shown in FIG. 6, in the comparative example, even if the Gap is changed, it is difficult to reduce the uniformity of the thickness of the organic film to less than 10%. On the other hand, in this embodiment, by changing the Gap, it is possible to reduce the uniformity of the thickness of the organic film to less than 10%. Accordingly, in this embodiment, it is possible to improve the uniformity of the thickness of the organic film formed on the substrate W.

The first embodiment has been described above. As described above, the substrate processing apparatus 10 according to this embodiment includes the processing container 209, the stage 211, the gas supply 230, and the elevating mechanism 240. The stage 211 is provided inside the processing container 209, and the substrate W is placed thereon. The gas supply 230 is provided at a position facing the stage 211, and is configured to supply a first processing gas containing a first monomer and a second processing gas containing a second monomer into the processing container to form a polymer film composed of the first monomer and the second monomer on the substrate W. The driver moves the stage 211 to change the distance between the gas supply 230 and the stage 211. Further, when viewed in the direction from the gas supply 230 toward the stage 211, the gas supply 230 supplies the first processing gas and the second processing gas into the space between the gas supply 230 and the stage 211 from the outside of the region on the stage 211 on which the substrate W is placed. Accordingly, it is possible to improve the uniformity of the thickness of the organic film formed on the substrate W.

In the embodiment described above, the discharge ports 232a discharge the first processing gas inward from the outside of the region on the stage 211 in which the substrate W is placed, when viewed in the direction from the gas supply 230 to the stage 211. The discharge ports 232b discharges the second processing gas inward from the outside of the region on the stage 211 in which the substrate W is placed, when viewed in a direction from the gas supply 230 to the stage 211. Thus, when the distance between the gas supply 230 and the stage 211 is short, the thickness of the organic film near the center of the substrate W can be made smaller than that near the edge of the substrate W. Further, when the distance between the gas supply 230 and the stage 211 is long, the thickness of the organic film near the center of the substrate W can be made larger than that near the edge of the substrate W.

Moreover, in the embodiment described above, the first monomer is an isocyanate, the second monomer is an amine, and the polymer formed on the substrate W contains a urea bond. The thickness of the polymer organic film formed on the substrate W is affected by the concentrations of the first monomer gas and the second monomer gas on the substrate W. In this embodiment, by adjusting the distance between the gas supply 230 and the stage 211, the ratio of the concentrations of the first monomer gas and the second monomer gas can be adjusted near the center and the edge of the substrate W. Accordingly, it is possible to improve the uniformity of the thickness of the organic film formed on the substrate W.

A part of the first monomer discharged from the discharge ports 232a into the processing space S_{P} may enter the discharge ports 232b, so that an organic film may be formed within the discharge ports 232b. Similarly, a part of the second monomer discharged from the discharge ports 232b into the processing space S_{P} may enter the discharge ports 232a, so that an organic film may be formed within the discharge ports 232a. When an organic film is formed within the discharge ports 232a and 232b, the discharge ports 232a and 232b may be blocked. A portion of the organic film formed at the discharge ports 232a and 232b can be removed by the active species supplied from the remote plasma generator 250 into the processing space S_{P}. However, since the spaces within the discharge ports 232a and 232b are small, the active species supplied into the processing space S_{P} may not be able to sufficiently enter deep into the discharge ports 232a and 232b. If the active species cannot sufficiently enter deep into the discharge ports 232a and 232b, it is difficult to sufficiently remove the organic film formed at the discharge ports 232a and 232b.

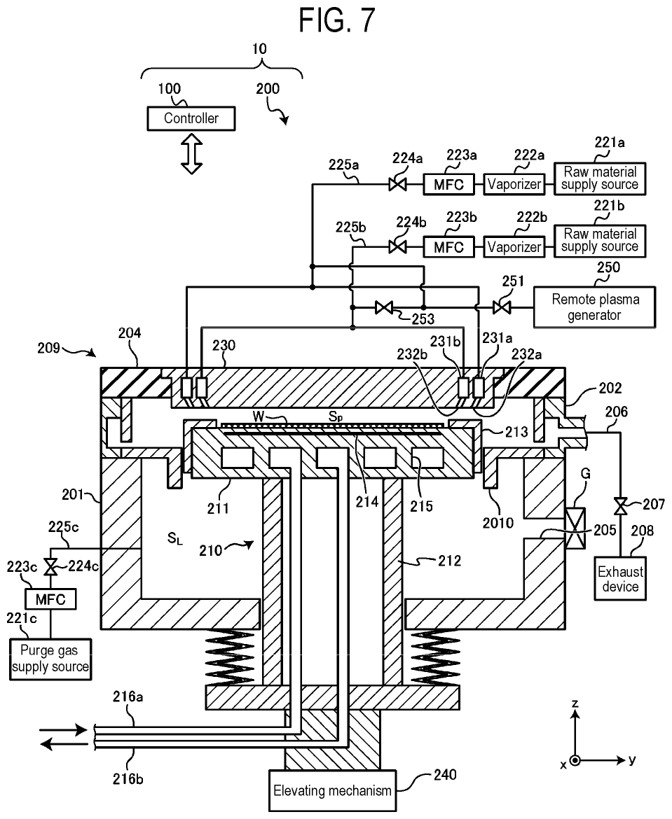

Therefore, as shown in FIG. 7, for example, the active species generated by the remote plasma generator 250 may be supplied to the pipe 225a via the valve 251, and may be supplied to the pipe 225b via the valve 251 and the valve 253. Thus, the organic film formed within the discharge ports 232a and 232b can be efficiently removed by the active species generated by the remote plasma generator 250. By providing the valve 253, it is possible to prevent the first monomer and the second monomer from being mixed in a pipe.

### Second Embodiment

In the first embodiment, the first monomer and the second monomer are individually discharged into the processing space S_{P} and mixed within the processing space S_{P}. In contrast, in this embodiment, a shower plate having a plurality of through holes is provided between the gas supply 230 and the stage 211. In this embodiment, the first monomer and the second monomer are mixed in the space between the gas supply 230 and the shower plate and then supplied into the processing space S_{P}. This makes it possible to further improve the uniformity of the thickness of the organic film formed on the substrate W. The following description will focus on the differences from the first embodiment.

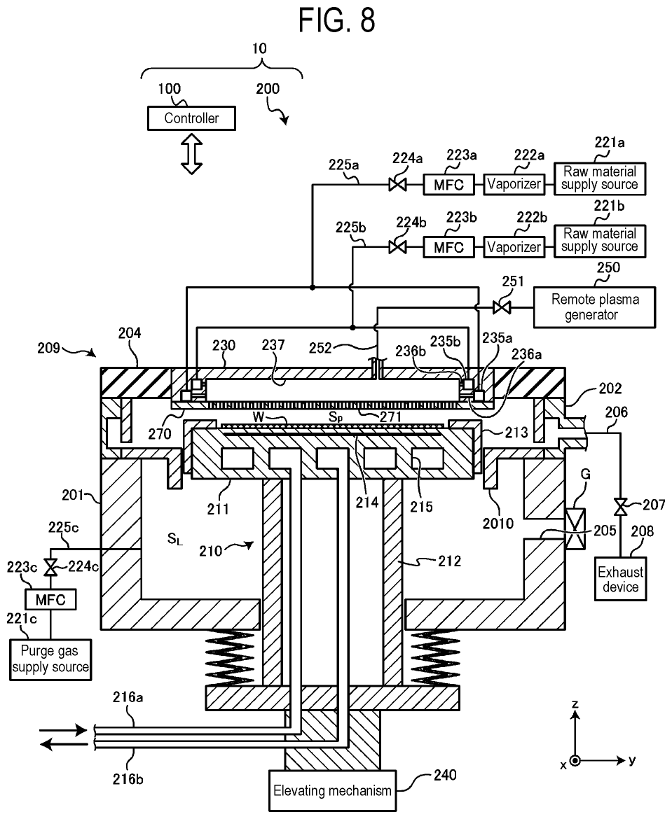

FIG. 8 is a schematic cross-sectional view showing an example of the substrate processing apparatus 10 according to the second embodiment of the present disclosure. The members designated by the same reference numerals in FIG. 8 as those shown in FIG. 1 have the same functions as the members described in FIG. 1, and therefore, redundant explanations thereof will be omitted.

The gas supply 230 according to this embodiment includes an annular diffusion chamber 235a and an annular diffusion chamber 235b, and a recess 237 formed inward the diffusion chamber 235a and the diffusion chamber 235b. The diffusion chamber 235a and diffusion chamber 235b do not communicate with each other and form independent spaces. A pipe 225a is connected to the diffusion chamber 235a, and a pipe 225b is connected to the diffusion chamber 235b. A shower plate 270 in which a plurality of through holes 271 are formed is provided on the lower surface of the gas supply 230.

A plurality of discharge ports 236a communicating with the diffusion chamber 235a and the recess 237 are formed between the diffusion chamber 235a and the recess 237, and a plurality of discharge ports 236b communicating with the diffusion chamber 235b and the recess 237 are formed between the diffusion chamber 235b and the recess 237. In this embodiment, each of the discharge ports 236a and 236b is formed to discharge a gas in a direction extending along the surface of the shower plate 270 (e.g., the xy plane) inward from the outside of the region of the stage 211 in which the substrate W is placed.

The isocyanate vapor (first monomer gas) supplied into the diffusion chamber 235a through the pipe 225a is diffused within the diffusion chamber 235a and is discharged into the space between the recess 237 and the shower plate 270 through the respective discharge ports 236a. Further, the amine vapor (second monomer gas) supplied into the diffusion chamber 235b via the pipe 225b is diffused within the diffusion chamber 235b and is discharged into the space between the recess 237 and the shower plate 270 through the discharge ports 236b. The discharge ports 236a are an example of first discharge ports, and the discharge ports 236b are an example of second discharge ports.

After the isocyanate vapor and the amine vapor are discharged into the space between the recess 237 and the shower plate 270 through the discharge ports 236a and the discharge ports 236b, they are sufficiently mixed in the space between the recess 237 and the shower plate 270. The gases mixed in the space between the recess 237 and the shower plate 270 are discharged into the processing space S_{P} through the through holes 271 of the shower plate 270 in the form of a shower. This makes it possible to improve the uniformity of the thickness of the organic film formed on the substrate W on the stage 211.

Further, in this embodiment, the remote plasma generator 250 is connected to the recess 237 of the gas supply 230 via a pipe 252 and a valve 251. Active species generated by the remote plasma generator 250 are supplied into the space between the recess 237 and the shower plate 270 via the valve 251 and the pipe 252. Thus, the organic film formed on the recess 237 and the shower plate 270 by the isocyanate vapor and the amine vapor can be efficiently removed. Furthermore, the active species supplied into the space between the recess 237 and the shower plate 270 are also supplied into the processing space S_{P} via the through holes 271. Accordingly, it is possible to efficiently remove the organic film adhering to the through holes 271, the side wall of the processing container 209, and the like.

For example, as shown in FIG. 7, the active species generated by the remote plasma generator 250 may be supplied to the pipe 225a via the valve 251, and may be supplied to the pipe 225b via the valve 251 and the valve 253. Accordingly, the organic film formed inside the discharge ports 236a and 236b can be efficiently removed by the active species generated by the remote plasma generator 250.

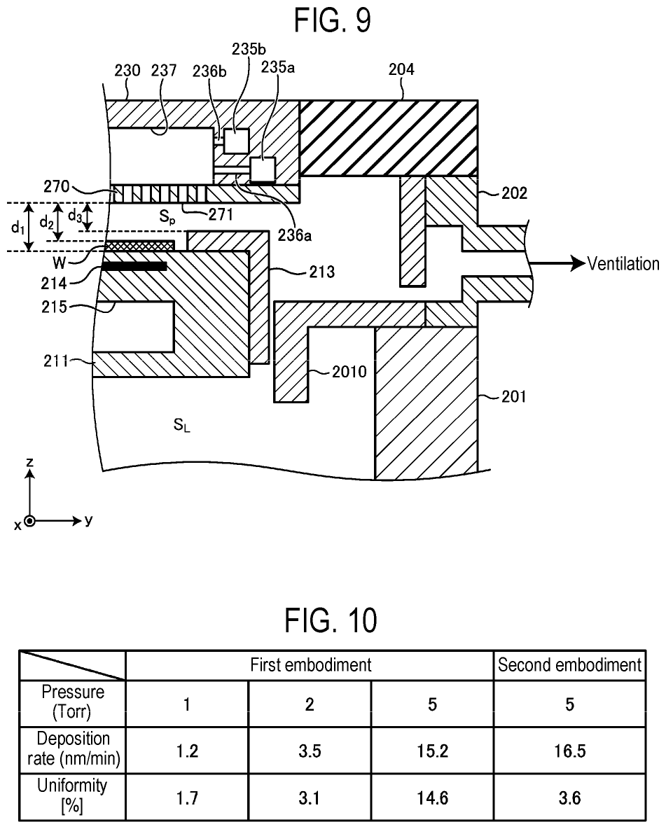

FIG. 9 is an enlarged sectional view for explaining an example of the distance between the stage 211 and the gas supply 230. The distance between the shower plate 270 and the stage 211 is defined as d₁, the distance between the shower plate 270 and the substrate W placed on the stage 211 is defined as d₂, and the distance between the shower plate 270 and the cover member 213 is defined as d₃.

In the process of forming the organic film on the substrate W, the distance d₁ is set to, for example, 5 mm to 30 mm, the distance d₂ is set to, for example, 4 mm to 29 mm (assuming the thickness of the substrate W is 1 mm), and the distance d₃ is set to, for example, 3 mm to 10 mm. In one example, the distance d₁ is, for example, 8.8 mm, the distance d₂ is, for example, 7.8 mm, and the distance d₃ is, for example, 5.2 mm. The distance d₂ is the value obtained by subtracting the thickness of the substrate W from the distance d₁. Further, the distance d₂ defines the uniformity of film formation, and the distance d₃ is a value that defines the uniformity of evacuation of the processing space S_{P}. Therefore, the distances d₂ and d₃ are set independently from each other according to their respective performance requirements. In this case, the distance d₃ is adjusted separately from the distance d₂, for example, by adjusting the thickness of the cover member 213. The distance d₃ may be adjusted separately from the distance d₂ by providing an annular protrusion that protrudes toward the cover member 213 on the edge portion of the shower plate 270 and adjusting the height of the protrusion.

### Deposition Rate and Uniformity

FIG. 10 is a diagram showing an example of the deposition rate and the uniformity of the thickness of the organic film. As the pressure inside the processing space S_{P} decreases, the gas diffusivity increases, thereby improving the uniformity. However, when the pressure inside the processing space S_{P} decreases, the concentration of gas inside the processing space S_{P} decreases, resulting in a decrease in the deposition rate.

For example, as shown in FIG. 10, in the first embodiment, as the pressure inside the processing space S_{P} decreases, the uniformity is improved, but the deposition rate is reduced. On the other hand, in the first embodiment, as the pressure inside the processing space S_{P} increases, the deposition rate is increased to thereby improve the throughput, but the uniformity is deteriorated. In contrast, in the second embodiment, it is possible to achieve both the high uniformity and the high deposition rate.

The second embodiment has been described above. As described above, the substrate processing apparatus 10 according to this embodiment further includes the shower plate 270 provided between the gas supply 230 and the stage 211. Further, the gas supply 230 has the discharge ports 232a and the discharge ports 232b. When viewed in the direction from the gas supply 230 toward the stage 211, the discharge ports 232a discharge the first processing gas into the space between the gas supply 230 and the stage 211 from the outside of the region on the stage 211 on which the substrate W is placed. When viewed in the direction from the gas supply 230 toward the stage 211, the discharge ports 232b discharge the second processing gas into the space between the gas supply 230 and the stage 211 from the outside of the region on the stage 211 on which the substrate W is placed. Accordingly, it is possible to further improve the uniformity of the thickness of the organic film formed on the substrate W.

Furthermore, in the second embodiment described above, the substrate processing apparatus 10 further includes a cleaner configured to supply active species into the space between the gas supply 230 and the shower plate 270. The cleaner includes a remote plasma generator 250 that converts a gas into plasma and supplies active species contained in the plasma into the space between the gas supply 230 and the shower plate 270. Accordingly, it is possible to efficiently remove the organic film adhering to the gas supply 230 and the processing container 209.

### Others

The technique disclosed herein is not limited to the embodiments described above, and may be modified in various forms within the scope of the gist thereof.

For example, the cleaner in each of the embodiments described above includes a remote plasma generator 250, and the active species generated by the remote plasma generator 250 are supplied into the processing space S_{P}, or into the processing space S_{P}, the pipe 225a and the pipe 225b. However, the disclosed technique is not limited thereto. The cleaner may supply a cleaning gas into the processing space S_{P}, or into the processing space S_{P}, the pipe 225a and the pipe 225b, thereby removing the organic films adhering to the side wall of the processing container 209, the insides of the discharge ports 232a and 232b, and the like. The cleaning gas is, for example, a fluorine-containing gas.

Furthermore, in each of the embodiments described above, the film of a polymer having a urea bond (—NH—CO—NH—) is formed on the surface of the substrate W by using isocyanate as the first monomer and amine as the second monomer. However, the disclosed technique is not limited thereto. For example, a film of a polymer having a 2-aminoethanol bond (—NH—CH₂—CH(OH)—) may be formed on the surface of the substrate W by using epoxide as the first monomer and amine as the second monomer. Alternatively, a film of a polymer having a urethane bond (—NH—CO—O—) may be formed on the surface of the substrate W by using isocyanate as the first monomer and alcohol as the second monomer. Alternatively, a film of a polymer having an amide bond (—NH—CO—) may be formed on the surface of the substrate W by using acyl halide as the first monomer and amine as the second monomer. Alternatively, a film of a polymer having an imide bond (—CO—N(—)—CO—) may be formed on the surface of the substrate W by using carboxylic acid anhydride as the first monomer and amine as the second monomer.

When the film of a polymer having an imide bond is formed on the surface of the substrate W, for example, pyromellitic dianhydride (PMDA) or the like may be used as the first monomer. Further, when the film of a polymer having an imide bond is formed on the surface of the substrate W, for example, 4,4′-oxydianiline (44ODA) or hexamethylenediamine (HMDA) may be used as the second monomer.

Further, in the above-described embodiments, a film forming apparatus is described as the substrate processing apparatus 10 by way of example. However, the disclosed technique is not limited thereto. As long as the distribution of gas in the processing container 209 affects the quality of processing on the substrate W, the disclosed technique may also be applied to an apparatus for performing etching, an apparatus for modifying the substrate W, or the like, in addition to the film forming apparatus. According to the present disclosure in some embodiments, it is possible to improve the uniformity of the thickness of an organic film formed on a substrate.

While certain embodiments have been described, these embodiments have been presented by way of example only, and are not intended to limit the scope of the disclosures. Indeed, the embodiments described herein may be embodied in a variety of other forms. Furthermore, various omissions, substitutions and changes in the form of the embodiments described herein may be made without departing from the spirit of the disclosures. The accompanying claims and their equivalents are intended to cover such forms or modifications as would fall within the scope and spirit of the disclosures.

Further, regarding the above-described embodiments, the following supplementary notes are further disclosed.

### Supplementary Note 1

A substrate processing apparatus comprising:
a processing container;
a stage on which a substrate is placed, the stage being provided in the processing container;
a gas supply provided at a position facing the stage and configured to supply a first processing gas containing a first monomer and a second processing gas containing a second monomer into the processing container to form a film of a polymer composed of the first monomer and the second monomer on the substrate; and
a driver configured to move the stage so as to change a distance between the gas supply and the stage,
wherein the gas supply is configured to supply the first processing gas and the second processing gas into a space between the gas supply and the stage from an outside of a region on the stage in which the substrate is placed, when viewed in a direction from the gas supply toward the stage.

### Supplementary Note 2

The apparatus of Supplementary Note 1, further comprising:
a shower plate provided between the gas supply and the stage,
wherein the gas supply includes a first discharge port configured to supply the first processing gas into the space between the gas supply and the stage from the outside of the region on the stage in which the substrate is placed, when viewed in the direction from the gas supply toward the stage, and a second discharge port configured to supply the second processing gas into the space between the gas supply and the stage from the outside of the region on the stage in which the substrate is placed, when viewed in the direction from the gas supply toward the stage.

### Supplementary Note 3

The apparatus of Supplementary Note 2, wherein the first discharge port is configured to supply the first processing gas inward from the outside of the region on the stage in which the substrate is placed, when viewed in the direction from the gas supply toward the stage, and the second discharge port is configured to supply the second processing gas inward from the outside of the region on the stage in which the substrate is placed, when viewed in the direction from the gas supply toward the stage.

### Supplementary Note 4

The apparatus of Supplementary Note 2 or 3, further comprising:
a cleaner configured to supply a cleaning gas or active species into a space between the gas supply and the shower plate.

### Supplementary Note 5

The apparatus of Supplementary Note 4, wherein the cleaner is configured to supply the cleaning gas or the active species into the space between the gas supply and the shower plate through the first discharge port and the second discharge port by supplying the cleaning gas or the active species to a first pipe for supplying the first processing gas into the first discharge port and to a second pipe for supplying the second processing gas into the second discharge port.

### Supplementary Note 6

The apparatus of Supplementary Note 4 or 5, wherein the cleaner includes a remote plasma generator configured to convert a gas into plasma and supply active species contained in the plasma into the space between the gas supply and the shower plate.

### Supplementary Note 7

The apparatus of any one of Supplementary Notes 1 to 6, wherein the first monomer is isocyanate, the second monomer is amine, and the polymer formed on the substrate contains a urea bond.

### Supplementary Note 8

The apparatus of any one of Supplementary Notes 1 to 6, wherein the first monomer is carboxylic acid anhydride, the second monomer is amine, and the polymer formed on the substrate contains an imide bond.

### Supplementary Note 9

The apparatus of any one of Supplementary Notes 1 to 6, wherein the first monomer is epoxide, the second monomer is amine, and the polymer formed on the substrate contains a 2-aminoethanol bond.

### Supplementary Note 10

The apparatus of any one of Supplementary Notes 1 to 6, wherein the first monomer is isocyanate, the second monomer is alcohol, and the polymer formed on the substrate contains a urethane bond.

### Supplementary Note 11

The apparatus of any one of Supplementary Notes 1 to 6, wherein the first monomer is acyl halide, the second monomer is amine, and the polymer formed on the substrate contains an amide bond.

What is claimed is:

1. A substrate processing apparatus comprising:
a processing container;
a stage on which a substrate is placed, the stage being provided in the processing container;
a gas supply provided at a position facing the stage and configured to supply a first processing gas containing a first monomer and a second processing gas containing a second monomer into the processing container to form a film of a polymer composed of the first monomer and the second monomer on the substrate; and
a driver configured to move the stage so as to change a distance between the gas supply and the stage,
wherein the gas supply is configured to supply the first processing gas and the second processing gas into a space between the gas supply and the stage from an outside of a region on the stage in which the substrate is placed, when viewed in a direction from the gas supply toward the stage.

2. The apparatus of claim 1, further comprising:
a shower plate provided between the gas supply and the stage,
wherein the gas supply includes a first discharge port configured to supply the first processing gas into the space between the gas supply and the stage from the outside of the region on the stage in which the substrate is placed, when viewed in the direction from the gas supply toward the stage, and a second discharge port configured to supply the second processing gas into the space between the gas supply and the stage from the outside of the region on the stage in which the substrate is placed, when viewed in the direction from the gas supply toward the stage.

3. The apparatus of claim 2, wherein the first discharge port is configured to supply the first processing gas inward from the outside of the region on the stage in which the substrate is placed, when viewed in the direction from the gas supply toward the stage, and the second discharge port is configured to supply the second processing gas inward from the outside of the region on the stage in which the substrate is placed, when viewed in the direction from the gas supply toward the stage.

4. The apparatus of claim 2, further comprising:
a cleaner configured to supply a cleaning gas or active species into a space between the gas supply and the shower plate.

5. The apparatus of claim 4, wherein the cleaner is configured to supply the cleaning gas or the active species into the space between the gas supply and the shower plate through the first discharge port and the second discharge port by supplying the cleaning gas or the active species to a first pipe for supplying the first processing gas into the first discharge port and to a second pipe for supplying the second processing gas into the second discharge port.

6. The apparatus of claim 4, wherein the cleaner includes a remote plasma generator configured to convert a gas into plasma and supply the active species contained in the plasma into the space between the gas supply and the shower plate.

7. The apparatus of claim 1, wherein the first monomer is isocyanate, the second monomer is amine, and the polymer formed on the substrate contains a urea bond.

8. The apparatus of claim 1, wherein the first monomer is carboxylic acid anhydride, the second monomer is amine, and the polymer formed on the substrate contains an imide bond.

9. The apparatus of claim 1, wherein the first monomer is epoxide, the second monomer is amine, and the polymer formed on the substrate contains a 2-aminoethanol bond.

10. The apparatus of claim 1, wherein the first monomer is isocyanate, the second monomer is alcohol, and the polymer formed on the substrate contains a urethane bond.

11. The apparatus of claim 1, wherein the first monomer is acyl halide, the second monomer is amine, and the polymer formed on the substrate contains an amide bond.

12. The apparatus of claim 3, further comprising:
a cleaner configured to supply a cleaning gas or active species into a space between the gas supply and the shower plate.

13. The apparatus of claim 12, wherein the cleaner is configured to supply the cleaning gas or the active species into the space between the gas supply and the shower plate through the first discharge port and the second discharge port by supplying the cleaning gas or the active species to a first pipe for supplying the first processing gas into the first discharge port and to a second pipe for supplying the second processing gas into the second discharge port.

14. The apparatus of claim 12, wherein the cleaner includes a remote plasma generator configured to convert a gas into plasma and supply the active species contained in the plasma into the space between the gas supply and the shower plate.
